(19) United States
(12) Patent Application Publication
(10) Pub. No.: US 2007/0179180 A1
(43) Pub. Date: Aug. 2, 2007

(54) Novel m3 muscarinic acetylcholine receptor antagonists

(75) Inventors: Jakob Busch-Petersen, King of Prussia, PA (US); Jian Jin, King of Prussia, PA (US); Michael R. Palovich, Collegeville, PA (US); Wei Fu, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 10/581,229

(22) PCT Filed: Dec. 3, 2004
(86) PCT No.: PCT/US04/40668

§ 371 Date: Mar. 15, 2007

Related U.S. Application Data
(60) Provisional application No. 60/526,766, filed on Dec. 3, 2003.

(51) Int. Cl. A61K 31/4709; A61K 31/454; A61K 31/4545; A61K 31/453; A61K 31/452; C07D 409/02; C07D 405/02; C07D 403/02; C07D 401/02

(52) U.S. Cl. 514/314; 514/323; 514/322; 514/321; 514/326; 514/319; 546/176; 546/194; 546/196; 546/198; 546/205; 546/207; 546/209

(57) ABSTRACT

Muscarinic Acetylcholine receptor antagonists and methods of using them are provided.

### FIELD OF THE INVENTION

This invention relates to novel derivatives of cyclic quaternary ammonium salts, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs, and these receptors can mediate both inhibitory and excitatory actions. For example, in smooth muscle found in the airways, bladder and gastrointestinal tract, M₃ mAChRs mediate contractile responses. For review, please see {Brown 1989 247/id}.

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mAChRs{Costello, Evans, et al. 1999 72/id}{Minette, Lammers, et al. 1989 248/id}. Similarly, inflammation of the gastrointestinal tract in inflammatory bowel disease (IBD) results in M₃ mAChR-mediated hypermotility {Oprins, Meijer, et al. 2000 245/id}. Incontinence due to bladder hypercontractility has also been demonstrated to be mediated through increased stimulation of M₃ mAChRs {Hegde & Eglen 1999 251 /id}. Thus the identification of subtytpe-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### SUMMARY OF THE INVENTION

This invention relates to compounds of Formula I 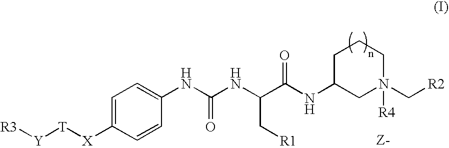
wherein

n is 0 or 1;

When X is nitrogen or oxygen, Y is nothing;

When Y is nitrogen or oxygen, X is nothing;

T is a sulfonyl group (SO2) or carbonyl group (CO);

When T=CO, X is oxygen or nitrogen;

Z⁻ is selected from the group consisting of halo, CF3COO⁻, mesylate, tosylate, or any other pharmaceutically acceptable counter ion;

R1 is selected from the group consisting of C₁-C₈ branched or unbranched alkyl, C₃-C₈ cycloalkyl, C₃-C₈ cycloalkyl lower alkyl, C₃-C₈ alkenyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl, C₃-C₈ cycloalkyl lower alkyl, phenyl and phenyl C1-C3 lower alkyl.

R2 is selected from the group consisting of C₁-C₈ branched or unbranched alkyl, C₃-C₈ cycloalkyl, C₃-C₈ cycloalkyl lower alkyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl and C₃-C₈ cycloalkyl lower alkyl and heterocycle rings;

R3 is selected from the group consisting of an unsubstituted or substituted following group: phenyl, phenyl C1-C6 lower alkyl, thiophenyl, thiophenyl C1-C6 lower alkyl, furanyl, furanyl C1-C6 lower alkyl, pyridinyl, pyridinyl C1-C6 lower alkyl, imidazolyl, imidazolyl C1-C6 lower alkyl, naphthyl, naphthyl C1-C6 lower alkyl, quinolinyl, quinolinyl C1-C6 lower alkyl, indolyl, indolyl C1-C6 lower alkyl, benzothiophenyl, benzothiophenyl C1-C6 lower alkyl, benzofuranyl, benzofuranyl C1-C6 lower alkyl, benzoimidazolyl, benzoimidazolyl C1-C6 lower alkyl, C₁-C₈ branched or unbranched alkyl, C₃-C₈ cycloalkyl, C₃-C₈ cycloalkyl C₁-C₆ lower alkyl, or C₃-C₈ alkenyl; wherein, when substituted, a group is substituted by one or more radicals selected from the group consisting of C₁-C₈ alkoxy, phenoxy, phenyl C₁-C₃ alkoxy, halo, hydroxy, amino, cyano, trifluoromethyl, methylenedioxy, ethylenedioxy, propylenedioxy, butylenedioxy, C₁-C₈ branched or unbranched alkyl, C₃-C₈ cycloalkyl, C₃-C₈ cycloalkyl lower alkyl, phenyl, phenyl C1-C3 lower alkyl, thiophenyl, thiophenyl C1-C3 lower alkyl, furanyl, furanyl C1-C3 lower alkyl, pyridinyl, pyridinyl C1-C3 lower alkyl, naphthyl, naphthyl C1-C3 lower alkyl, quinolinyl, quinolinyl C1-C3 lower alkyl, indolyl, indolyl C1-C3 lower alkyl, benzothiophenyl, benzothiophenyl C1-C3 lower alkyl, benzofuranyl, benzofuranyl C1-C3 lower alkyl, COOH, COR6, COOR6, CONHR6, CON(R6)2, COG, NHR6, N(R6)2, G, OCOR6, OCONHR6, NHCOR6, N(R6)COR6, NHCOOR6 and NHCONHR6;

R4 is selected from the group consisting of C₁-C₈ branched or unbranched alkyl, C₃-C₈ cycloalkyl, C₃-C₈ cycloalkyl lower alkyl.

### DETAILED DESCRIPTION

The present invention includes all hydrates, solvates, complexes and prodrugs of the compounds of this invention. Prodrugs are any covalently bonded compounds that release the active parent drug according to Formula I—in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

The term “C₁-C₈ alkyl” and “C₁-C₆ alkyl” is used herein includes both straight or branched chain radicals of 1 to 6 or 8 carbon atoms. By example this term includes, but is not limited to methyl, ethyl, n-propyl, isopropyl, n-butyl, sec-butyl, isobutyl, tert-butyl, pentyl, hexyl, heptyl, octyl and the like. “Lower alkyl” has the same meaning as C₁-C₈ alkyl.

Herein “C₁-C₈ alkoxy” includes straight and branched chain radicals of the likes of —O—CH₃, —O—CH₂CH₃, and the n-propoxy, isopropoxy, n-butoxy, sec-butoxy, isobutoxy, tert-butoxy, pentoxy, and hexoxy, and the like.

“C₃-C₈-cycloalkyl” as applied herein is meant to include substituted and unsubstituted cyclopropane, cyclobutane, cyclopentane and cyclohexane, and the like.

“Halogen” or “halo” means F, Cl, Br, and I.

The preferred compounds of Formula I include those compounds wherein:

n is 0 or 1;

When X is nitrogen or oxygen, Y is nothing;

When Y is nitrogen or oxygen, X is nothing;

T is sulfonyl group SO2 or conbonyl group CO;

R1 is selected from the group consisting of C₁-C₈ branched or unbranched alkyl, C₃-C₈ cycloalkyl, C₃-C₈ cycloalkyl lower alkyl, C₃-C₈ alkenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl, C₃-C₈ cycloalkyl lower alkyl, phenyl and phenyl C1-C3 lower alkyl; or R2 and R3 is —(CH₂)ⱼ—, or —(CH₂)ᵢ-Phenyl-(CH₂)ᵢ—; wherein, j is an interger from 3 to 8; i is an integer from 1 to 3.

R2 is selected from the group consisting of hydrogen, hydroxy, amino, halo, cyano, trifluoromethyl, C₁-C₈ alkoxy, C₁-C₈ alkyl, C₃-C₈ cycloalkyl, C₃-C₈ cycloalkyl lower alkyl, phenyl, phenyl C1-C3 lower alkyl, phenylcarbonyl;

R3 is selected from the group consisting of an unsubstituted or substituted following group: phenyl C1-C6 lower alkyl, thiophenyl C1-C6 lower alkyl, furanyl C1-C6 lower alkyl, pyridinyl C1-C6 lower alkyl, imidazolyl C1-C6 lower alkyl, naphthyl C1-C6 lower alkyl, quinolinyl C1-C6 lower alkyl, indolyl C1-C6 lower alkyl, benzothiophenyl C1-C6 lower alkyl, benzofuranyl C1-C6 lower alkyl, benzoimidazolyl C1-C6 lower alkyl, C₁-C₈ branched or unbranched alkyl, C₃-C₈ cycloalkyl, C₃-C₈ cycloalkyl C₁-C₆ lower alkyl, or C₃-C₈ alkenyl; wherein, when substituted, a group is substituted by one or more radicals selected from the group consisting of C₁-C₈ alkoxy, phenoxy, phenyl C₁-C₃ alkoxy, halo, hydroxy, amino, cyano, trifluoromethyl, methylenedioxy, ethylenedioxy, propylenedioxy, butylenedioxy, C₁-C₈ branched or unbranched alkyl, C₃-C₈ cycloalkyl, C₃-C₈ cycloalkyl lower alkyl, phenyl, phenyl C1-C3 lower alkyl, thiophenyl, thiophenyl C1-C3 lower alkyl, furanyl, furanyl C1-C3 lower alkyl, pyridinyl, pyridinyl C1-C3 lower alkyl, naphthyl, naphthyl C1-C3 lower alkyl, quinolinyl, quinolinyl C1-C3 lower alkyl, indolyl, indolyl C1-C3 lower alkyl, benzothiophenyl, benzothiophenyl C1-C3 lower alkyl, benzofuranyl, benzofuranyl C1-C3 lower alkyl, COOH, COR6, COOR6, CONHR6, CON(R6)2, COG, NHR6, N(R6)2, G, OCOR6, OCONHR6, NHCOR6, N(R6)COR6, NHCOOR6 and NHCONHR6;

R4 is selected from the group consisting of C₁-C₈ branched or unbranched alkyl, C₃-C₈ cycloalkyl, C₃-C₈ cycloalkyl lower alkyl, or phenyl C1-C3 lower alkyl;

Even more preferred are those compounds where:

n is 1;

X is nitrogen or oxygen, Y is nothing;

T is sulfonyl group SO2;

Z⁻ is selected from the group consisting of I⁻, Br⁻, Cl⁻, F⁻, CF3COO⁻, mesylate, tosylate, or any other pharmaceutically acceptable counter ion;

R4 is selected from the group consisting of C₁-C₈ branched or unbranched alkyl, C₃-C₈ cycloalkyl, C₃-C₈ cycloalkyl lower alkyl,

R3 is selected from the group consisting of an unsubstituted or substituted following group: phenyl C1-C6 lower alkyl, thiophenyl C1-C6 lower alkyl, furanyl C1-C6 lower alkyl, pyridinyl C1-C6 lower alkyl, imidazolyl C1-C6 lower alkyl, naphthyl C1-C6 lower alkyl, quinolinyl C1-C6 lower alkyl, indolyl C1-C6 lower alkyl, benzothiophenyl C1-C6 lower alkyl, benzofuranyl C1-C6 lower alkyl, benzoimidazolyl C1-C6 lower alkyl, C₁-C₈ branched or unbranched alkyl, C₃-C₈ cycloalkyl, C₃-C₈ cycloalkyl C₁-C₆ lower alkyl, or C₃-C₈ alkenyl; wherein, when substituted, a group is substituted by one or more radicals selected from the group consisting of C₁-C₈ alkoxy, phenoxy, phenyl C₁-C₃ alkoxy, halo, hydroxy, amino, cyano, trifluoromethyl, methylenedioxy, ethylenedioxy, propylenedioxy, butylenedioxy, C₁-C₈ branched or unbranched alkyl, C₃-C₈ cycloalkyl, C₃-C₈ cycloalkyl lower alkyl, phenyl, phenyl C1-C3 lower alkyl, thiophenyl, thiophenyl C1-C3 lower alkyl, furanyl, furanyl C1-C3 lower alkyl, pyridinyl, pyridinyl C1-C3 lower alkyl, naphthyl, naphthyl C1-C3 lower alkyl, quinolinyl, quinolinyl C1-C3 lower alkyl, indolyl, indolyl C1-C3 lower alkyl, benzothiophenyl, benzothiophenyl C1-C3 lower alkyl, benzofuranyl, benzofuranyl C1-C3 lower alkyl, COOH, COR6, COOR6, CONHR6, CON(R6)2, COG, NHR6, N(R6)2, G, OCOR6 and NHCOR6;

R2 is selected from the group consisting of hydroxy, amino, halo, cyano, trifluoromethyl, C₁-C₈ alkoxy, C₁-C₈ alkyl, C₃-C₈ cycloalkyl, C₃-C₈ cycloalkyl lower alkyl, phenyl, phenyl C1-C3 lower alkyl, phenylcarbonyl;

R1 is selected from the group consisting of C₁-C₈ branched or unbranched alkyl, C₃-C₈ cycloalkyl, C₃-C₈ cycloalkyl lower alkyl, or C₃-C₈ alkenyl;

or R1 and R2 is —(CH₂)ⱼ—, or —(CH₂)ᵢ-Phenyl-(CH₂)ᵢ—.

The preferred compounds are selected from the group consisting of:
N-((3S)-1-{[3,4-bis(methyloxy)phenyl]methyl}-1-methyl-3-piperidiniumyl)-N-{[(4-{[(2 2,2-trifluoroethyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(5-methyl-2-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(4-methyl-2-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-[({4-[(8-quinolinylsulfonyl)oxy]phenyl}amino)carbonyl]-L-tyrosinamide trifluoroacetate;
N-((3S)-1-{[3,4-bis(methyloxy)phenyl]methyl}-1-methyl-3-piperidiniumyl)-N-({[4-({[3,4-bis(methyloxy)phenyl]sulfonyl}oxy)phenyl]amino}carbonyl)-L-tyrosinamide trifluoroacetate;
N-((3S)-1-{[3,4-bis(methyloxy)phenyl]methyl}-1-methyl-3-piperidiniumyl)-N-{[(4-{[(2-bromophenyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(4-fluorophenyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-[({4-[(phenylsulfonyl)oxy]phenyl}amino)carbonyl]-L-tyrosinamide trifluoroacetate;
N-((3S)-1-{[3,4-bis(methyloxy)phenyl]methyl}-1-methyl-3-piperidiniumyl)-N-{[(4-{[(5-bromo-2-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-[({4-[(3-thienylsulfonyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-{[(4-{[(2,5-dimethyl-3-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(2,2,2-trifluoroethyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(5-methyl-2-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(4-methyl-2-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(5-chloro-2-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-[({4-[(methylsulfonyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-[({4-[(propylsulfonyl)oxy]phenyl}amino)carbonyl]-L-tyrosinamide trifluoroacetate;
N-({[4-({[2-(acetylamino)-4-methyl-1,3-thiazol-5-yl]sulfonyl}oxy)phenyl]amino}carbonyl)-N-[(3S)-1-(1,3-benzodioxol-5-ylmethyl)-1-methyl-3-piperidiniumyl]-L-tyrosinamide trifluoroacetate;
N-[(3S)-1-(1,3-benzodioxol-5-ylmethyl)-1-methyl-3-piperidiniumyl]-N-({[4-({[4-(phenylsulfonyl)-2-thienyl]sulfonyl}oxy)phenyl]amino}carbonyl)-L-tyrosinamide trifluoroacetate;
N-[(3S)-1-(1,3-benzodioxol-5-ylmethyl)-1-methyl-3-piperidiniumyl]-N-{[(4-{[(5-chloro-2,1,3-benzoxadiazol-4-yl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-[({4-[(2-naphthalenylsulfonyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-{[(4-{[(2,2,2-trifluoroethyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(5-methyl-2-thienyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(4-methyl-2-thienyl)sulfonyl]oxy}phenyl)amino]carbonyl}-L-tyrosinamide trifluoroacetate;
N-{[(4-{[(4-cyanophenyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{(3S)-1-[(4-fluorophenyl)methyl]-1-methyl-3-piperidiniumyl}-N-({[4-({[4-(trifluoromethyl)phenyl]sulfonyl}oxy)phenyl]amino}carbonyl)-L-tyrosinamide trifluoroacetate;
N-{(3S)-1-[(4-fluorophenyl)methyl]-1-methyl-3-piperidiniumyl}-N-({[4-({[5-(3-isoxazolyl)-2-thienyl]sulfonyl}oxy)phenyl]amino}carbonyl)-L-tyrosinamide trifluoroacetate;
N-{(3S)-1-[(4-fluorophenyl)methyl]-1-methyl-3-piperidiniumyl}-N-{[(4-{[(3-fluorophenyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(1,3,5-trimethyl-1H-pyrazol-4-yl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(5-methyl-4-isoxazolyl)sulfonyl]oxy}phenyl)amino]carbonyl}-L-tyrosinamide trifluoroacetate;
N-{[(4-{[(3,5-dimethyl-4-isoxazolyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{[(4-{[(2,4-dichlorophenyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{(3S)-1-[(4-fluorophenyl)methyl]-1-methyl-3-piperidiniumyl}-N-[({4-[({4-[(trifluoromethyl)oxy]phenyl}sulfonyl)oxy]phenyl}amino)carbonyl]-L-tyrosinamide trifluoroacetate;
N-{(3S)-1-[(4-fluorophenyl)methyl]-1-methyl-3-piperidiniumyl}-N-{[(4-{[(1-methyl-1H-imidazol-4-yl)sulfonyl]oxy}phenyl)amino]carbonyl}-L-tyrosinamide trifluoroacetate;
N-[({4-[(cyclohexylcarbonyl)oxy]phenyl}amino)carbonyl]-N-{(3S)-1-[(4-fluorophenyl)methyl]-1-methyl-3-piperidiniumyl}-L-tyrosinamide trifluoroacetate;
N-[(3S)-1-(1,3-benzodioxol-5-ylmethyl)-1-methyl-3-piperidiniumyl]-N-[({4-[(cyclohexylcarbonyl)oxy]phenyl}amino)carbonyl]-L-tyrosinamide trifluoroacetate;
N-{(3S)-1-[(4-chlorophenyl)methyl]-1-methyl-3-piperidiniumyl}-N-[({4-[(cyclohexylcarbonyl)oxy]phenyl}amino)carbonyl]-L-tyrosinamide trifluoroacetate;
N-{(3S)-1-[(3-chlorophenyl)methyl]-1-methyl-3-piperidiniumyl}-N-[({4-[(cyclohexylcarbonyl)oxy]phenyl}amino)carbonyl]-L-tyrosinamide trifluoroacetate;
N-((3S)-1-{[3,4-bis(methyloxy)phenyl]methyl}-1-methyl-3-piperidiniumyl)-N-[({4-[(cyclohexylcarbonyl)oxy]phenyl}amino)carbonyl]-L-tyrosinamide trifluoroacetate;
N-{(3S)-1-[(3-hydroxyphenyl)methyl]-1-methyl-3-piperidiniumyl}-N-[({4-[(2-methylpropanoyl)oxy]phenyl}amino)carbonyl]-L-tyrosinamide trifluoroacetate;
N-{(3S)-1-[(3-chlorophenyl)methyl]-1-methyl-3-piperidiniumyl}-N-[({4-[(2-methylpropanoyl)oxy]phenyl}amino)carbonyl]-L-tyrosinamide trifluoroacetate;
N-{(3S)-1-[(4-chlorophenyl)methyl]-1-methyl-3-piperidiniumyl}-N-[({4-[(2-methylpropanoyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-{[(4-{[(1-methylethyl)amino]sulfonyl}phenyl)amino]carbonyl}-L-tyrosinamide trifluoroacetate;
N-{(3S)-1-ethyl-1-[(3-hydroxyphenyl)methyl]-3-pyrrolidiniumyl}-N-{[(4-{[(1-methylethyl)amino]sulfonyl}phenyl)amino]carbonyl}-L-tyrosinamide trifluoroacetate;
   or any other pharmaceutically acceptable salt.

The most preferred compounds are selected from the group consisting of:
N-((3S)-1-{[3,4-bis(methyloxy)phenyl]methyl}-1-methyl-3-piperidiniumyl)-N-{[(4-{[(2,5-dimethyl-3-thienyl)sulfonyl]oxy}phenyl)amino]carbonyl}-L-tyrosinamide trifluoroacetate
N-((3S)-1-{[3,4-bis(methyloxy)phenyl]methyl}-1-methyl-3-piperidiniumyl)-N-{[(4-{[(2,5-dimethyl-3-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(1-methylethyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(6-chloro-3-methyl-1-benzothien-2-yl)sulfonyl]oxy}phenyl)amino]carbonyl}-L-tyrosinamide trifluoroacetate;
N-{[(4-{[(2,5-dimethyl-3-thienyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{(3S)-1-[(4-fluorophenyl)methyl]-1-methyl-3-piperidiniumyl}-N-{[(4-{[(1-methylethyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(1-methylethyl)sulfonyl]oxy}phenyl)amino]carbonyl}-L-tyrosinamide trifluoroacetate;
N-{(3S)-1-[(3-hydroxyphenyl)methyl]-1-methyl-3-piperidiniumyl}-N-{[(4-{[(1-methylethyl)amino]sulfonyl}phenyl)amino]carbonyl}-L-tyrosinamide trifluoroacetate

### Methods of Preparation

Preparation

The compounds of Formula (I) may be obtained by applying synthetic procedures, some of which are illustrated in the Schemes below. The synthesis provided for these Schemes is applicable for producing compounds of Formula (I) having a variety of different R1, R2, R3 and R4, which are reacted, employing substituents which are suitable protected, to achieve compatibility with the reactions outlined herein. Subsequent deprotection, in those cases, then affords compounds of the nature generally disclosed. While some Schemes are shown with specific compounds, this is merely for illustration purpose only.

Preparation 1

Resin-bound amines 3 were prepared by reductive alkylation of 2,6-dimethoxy-4-polystyrenebenzyloxy-benzaldehyde (DMHB resin) with N-protected diamine HCl salts 2, which were prepared from Boc-protected diamines 1 (Scheme 1). Reactions of 3 with Fmoc-protected amino acids, followed by removal of the protecting group, provided resin-bound intermediates 4. 4-Hydroxyl anline was coupled with resin-bound intermediates 4 to afford the corresponding resin-bound urea 5, which was subsequently treated with potassium carbonate and thiophenol to give secondary amines. Reductive amination of secondary amine with aldehydes produced resin-bound tertiary amines 6. Amines 6 were then reacted with a series of sulfonyl chlorides to give the corresponding resin-bound sulfonyl esters 7, which were treated with alkyl halides(R4Z) to give the corresponding resin-bound quaternary ammonium salts. Resin-bound quaternary ammonium salts were cleaved with 50% trifluoroacetic acid in dichloromethane to afford targeted compounds 8.

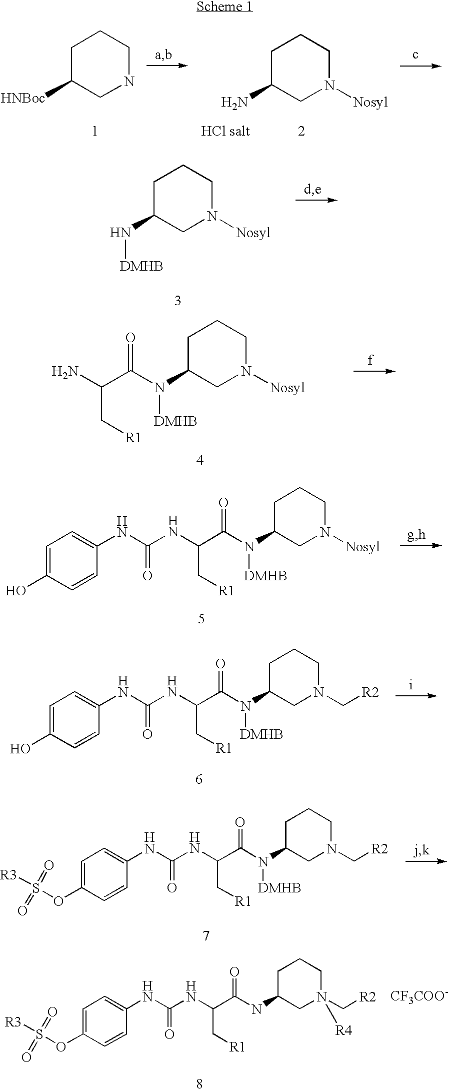

Conditions: a) 2-nitrobenzenesulfonyl chloride (Nosyl-Cl), pyridine, CH₂Cl₂, 0° C.—rt; b) 4 M HCl in 1,4-dioxane, MeOH, rt; c) 2,6-dimethoxy-4-polystyrenebenzyloxy-benzaldehyde (DMHB resin), Na(OAc)₃BH, diisopropylethylamine, 10% acetic acid in 1-methyl-2-pyrrolidinone, rt; d) Fmoc-protected amino acids, 1,3-diisopropylcarbodiimide, 1-hydroxy-7-azabenzotriazole, 1-methyl-2-pyrrolidinone, rt; e) 20% piperidine in 1-methyl-2-pyrrolidinone, rt; f) 4-nitrobenzene chloroformate, 4-hydroxyl aniline, tetrahydrofuran, diisopropylethylamine, dimethyl formamide, rt; g) K₂CO₃, PhSH, 1-methyl-2-pyrrolidinone, rt; h) R₂CHO, Na(OAc)₃BH, 10% acetic acid in 1-methyl-2-pyrrolidinone, rt; i) sulfonyl chloride, TEA, dichloromethane j) R₄Z, acetonitrile, rt; k) 50% trifluoroacetic acid in dichloromethane, rt. The following examples are provided as illustrative of the present invention but not limiting in any way:

### EXAMPLE 1

### Preparation of N-((3S)-1-{[3,4-bis(methyloxy)phenyl]methyl}-1-methyl-3-piperidiniumyl)-N-{[(4-{[(2,5-dimethyl-3-thienyl)sulfonyl]oxy}phenyl)amino]carbonyl}-L-tyrosinamide Trifluoroacetate

a) 3(S)-amino-N-(2-nitrobenzenesulfonyl)pyrrolidine HCl Salt

To a solution of 3(S)-(−)-(tert-butoxycarbonyl-amino)pyrrolidine (20.12 g, 108 mmol) in 250 mL of anhydrous methylene chloride at 0° C. was added 13.1 mL (162 mmol) of anhydrous pyridine, followed by slow addition of 25.2 g (113.4 mmol) of 2-nitrobenzenesulfonyl chloride. The mixture was warmed to rt over 1 h and stirred at rt for 16 h. The mixture was poured into 300 mL of 1 M aqueous NaHCO₃ solution. After the resulting mixture was stirred at rt for 30 min, the organic layer was separated and washed with 500 mL of 1N aqueous HCl solution twice. The resulting organic layer was dried over MgSO₄ and concentrated in vacuo. The residue was used for the the next step without further purification.

To a mixture of the above residue in 140 mL of anhydrous MeOH was added 136 mL (544 mmol) of 4 M HCl in 1,4-dioxane solution. The mixture was stirred at rt for 16 h, concentrated in vacuo and further dried in vaccum oven at 35° C. for 24 h to yield 3(S)-amino-N-(2-nitrobenzenesulfonyl)pyrrolidine HCl salt as a yellow solid (30.5 g, 92% over the two steps): ¹H NMR (400 MHz, d₆-DMSO) δ 8.63 (s, 3H), 8.08-7.98 (m, 2H), 7.96-7.83 (m, 2H), 3.88-3.77 (m, 1H), 3.66-3.56 (m, 2H), 3.46-3.35 (m, 2H), 2.28-2.16 (m, 1H), 2.07-1.96 (m, 1H).

b) DMHB resin bound O-(1,1-dimethylethyl)-N-{(3S)-1-[(2-nitrophenyl)sulfonyl]-3-pyrrolidinyl}-L-tyrosinamide

To a mixture of 7.20 g (10.37 mmol, 1.44 mmol/g) of 2,6-dimethoxy-4-polystyrenebenzyloxy-benzaldehyde (DMHB resin) in 156 mL of 10% acetic acid in anhydrous 1-methyl-2-pyrrolidinone was added 9.56 g (31.1 mmol) of 3(S)-amino-N-(2-nitrobenzenesulfonyl)pyrrolidine HCl salt and 9.03 mL (51.84 mmol) of diisopropylethyl amine, followed by addition of 11.0 g (51.84 mmol) of sodium triacetoxyborohydride. After the resulting mixture was shaken at rt for 72 h, the resin was washed with DMF (3×250 mL), CH₂Cl₂/MeOH (1:1, 3×250 mL) and MeOH (3×250 mL). The resulting resin was dried in vacuum oven at 35° C. for 24 h. Elemental analysis N: 4.16, S: 3.12.

To a mixture of 800 mg (0.860 mmol, 1.075 mmol/g) of the above resin in 15 mL of anhydrous 1-methyl-2-pyrrolidinone was added 1.98 g (4.30 mmol) of Fmoc-Try(tBu)-OH and 117 mg (0.86 mmol) of 1-hydroxy-7-azabenzotriazole, followed by addition of 0.82 mL (5.16 mmol) of 1,3-diisopropylcarbodiimide. After the resulting mixture was shaken at rt for 24 h, the resin was washed with DMF (3×25 mL), CH₂Cl₂/MeOH (1:1, 3×25 mL) and MeOH (3×25 mL). The resulting resin was dried in vacuum oven at 35° C. for 24 h. An analytical amount of resin was cleaved with 50% trifluoroacetic acid in dichloroethane for 2 h at rt. The resulting solution was concentrated in vacuo: MS (ESI) 657 [M+H-tBu]⁺.

The above resin (0.860 mmol) was treated with 15 mL of 20% piperidine in anhydrous 1-methyl-2-pyrrolidinone solution. After the mixture was shaken at rt for 15 min, the solution was drained and another 15 mL of 20% piperidine in anhydrous 1-methyl-2-pyrrolidinone solution was added. The mixture was shaken at rt for another 15 min. The solution was drained and the resin was washed with DMF (3×25 mL), CH₂Cl₂/MeOH (1:1, 3×25 mL) and MeOH (3×25 mL). The resulting resin was dried in vacuum oven at 35° C. for 24 h to afford DHMB resin bound O-(1,1-dimethylethyl)-N-{(3S)-1-[(2-nitrophenyl)sulfonyl]-3-pyrrolidinyl}-L-tyrosinamide (0.86 mmol). An analytical amount of resin was cleaved with 50% trifluoroacetic acid in dichloroethane for 2 h at rt. The resulting solution was concentrated in vacuo: MS (ESI) 435 [M+H-tBu]⁺.

c) N-((3S)-1-{[3,4-bis(methyloxy)phenyl]methyl}-1-methyl-3-piperidiniumyl)-N-{[(4-{[(2,5-dimethyl-3-thienyl)sulfonyl]oxy}phenyl)amino]carbonyl}-L-tyrosinamide

To a mixture of 1.1 g (9.26 mmol) of 4-hydroxylaniline in 20 ml anhydrous tetrahydrofuran was added 1.81 g (9.26 mmol) 4-nitrobenzenechloroformate. The reaction mixture was stirred at room temperature for half an hour and concentrated. Diisopropylethylamine (5 mL, 35.25 mmol), DMHB resin bound O-(1,1-dimethylethyl)-N-{(3S)-1-[(2-nitrophenyl)sulfonyl]-3-pyrrolidinyl}-L-tyrosinamide 4 (3 g, 2.4 mmol) and dimethyl formamide (25 mL) were added to reaction mixture and shaked overnight. The resin was washed with DMF (3×10 mL), CH₂Cl₂/MeOH (1:1, 3×10 mL) and MeOH (3×10 mL). The resulting urea resin 5 was dried in vacuum oven at 35° C. for 24 h. An analytical amount of resin was cleaved with 50% trifluoroacetic acid in dichloromethane for 2 h at rt. The resulting solution was concentrated in vacuo: MS (ESI) 584.4 [M+H-tBu]⁺.

To a mixture of urea resin 5 (2.4 mmol) in 60 mL of 1-methyl-2-pyrrolidinone was added 2.5 g (18 mmol) of K₂CO₃ and 0.92 mL (9 mmol) of PhSH. After the resulting mixture was shaken at rt for 2 h, the resin was washed with DMF (3×10 mL), H₂O (3×10 mL), DMF (3×10 mL), CH₂Cl₂/MeOH (1:1, 3×10 mL) and MeOH (3×10 mL). The resulting resin was dried in vacuum oven at 35° C. for 24 h. To a mixture of the above dry resin secondary amine 1 g (0.8 mmol) in 40 mL of 10% HOAc in anhydrous 1-methyl-2-pyrrolidinone solution was added 997 mg (6 mmol) of 3,4-bis(methyloxy)benzaldehyde and 1.272 g (6 mmol) of sodium triacetoxyborohydride. After the resulting mixture was shaken at rt for 72 h, the resin was washed with DMF (3×10 mL), CH₂Cl₂/MeOH (1:1, 3×10 mL) and MeOH (3×10 mL). The resulting resin 6 was dried in vacuum oven at 35° C. for 24 h. An analytical amount of resin was cleaved with 50% trifluoroacetic acid in dichloroethane for 2 h at rt. The resulting solution was concentrated in vacuo: MS (ESI) 550 [M+H-tBu]⁺.

To a mixture of resin-bound tertiary amines 6 (100 mg, 0.08 mmol) in 10 mL methylenechloride and triethyl amine (0.52 mL, 4 mmol) at 0° C. was added 2,5-dimethyl-3-thiophenesulfonyl chloride (421.4 mg, 2 mmol). The reaction mixture was warmed to room temperature and shaked overnight. The resin was washed with DMF (3×10 mL), CH₂Cl₂/MeOH (1:1, 3×10 mL), MeOH (3×10 mL) and CH₂Cl₂ (3×10 mL). The resulting resin was dried in vacuum oven at 35° C. for 24 h.

To a mixture of the above dry resin (0.08 mmol) in 3 mL of anhydrous acetonitrile was added 120 μL (1.918 mmol) of iodomethane. After the mixture was shaken at rt for 16 h, the resin was washed with DMF (3×10 mL), CH₂Cl₂/MeOH (1:1, 3×10 mL), MeOH (3×10 mL) and CH₂Cl₂ (3×10 mL). The resulting resin was dried in vacuum oven at 35° C. for 24 h. The dry resin was treated with 4 mL of 50% trifluoroacetic acid in dichloroethane at rt for 2 h. After the cleavage solution was collected, the resin was treated with another 4 mL of 50% trifluoroacetic acid in dichloroethane at rt for 10 min. The combined cleavage solutions were concentrated in vacuo. The residue was purified using a Gilson semi-preparative HPLC system with a YMC ODS-A (C-18) column 50 mm by 20 mm ID, eluting with 10% B to 90% B in 3.2 min, hold for 1 min where A=H₂O (0.1% trifluoroacetic acid) and B=CH₃CN (0.1% trifluoroacetic acid) pumped at 25 mL/min, to produce N-[({4-[(ethyloxy)carbonyl]phenyl}amino)carbonyl]-N-{(3S)-1-[(4-hydroxyphenyl)methyl]-1-methyl-3-pyrrolidiniumyl}-L-tyrosinamide trifluoroacetate (white powder, 32 mg, 54% over 6 steps): MS (ESI) 737.4 [M]⁺.

Proceeding in a similar manner as described in example 1, but replacing 2,5-dimethyl-3-thiophenesulfonyl chloride with the appropriate sulfonyl chlorides and/or replacing 3,4-bis(methyloxy)benzaldehyde with the appropriate aldehydes, the compounds listed in Tables 1-3 were prepared.

TABLE 1

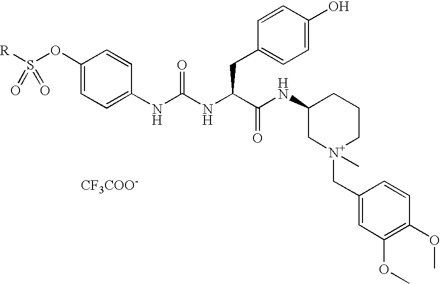

| Example | R | MS [M]⁺ |
|---|---|---|
| 1 | 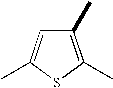 | 737 |
| 2 | 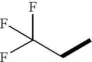 | 709 |
| 3 | 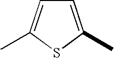 | 723 |
| 4 | 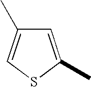 | 723 |
| 5 | 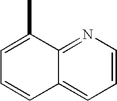 | 754 |
| 6 | 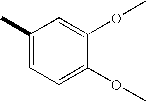 | 763 |
| 7 | 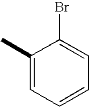 | 781 |
| 8 | 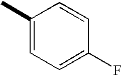 | 721 |
| 9 | 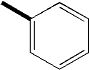 | 703 |
| 10 | 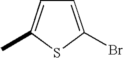 | 788 |
| 11 | 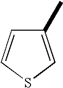 | 709 |
| 12 | 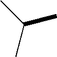 | 669 |

TABLE 2

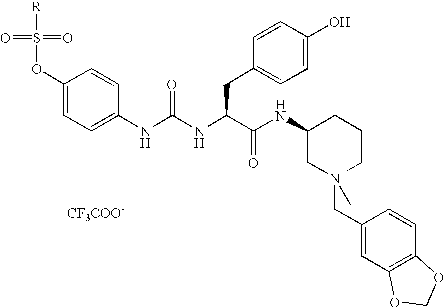

| Example | R | MS [M]⁺ |
|---|---|---|
| 13 | 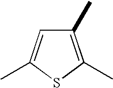 | 721 |
| 14 | 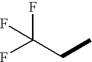 | 693 |
| 15 | 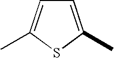 | 707 |
| 16 | 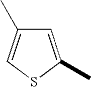 | 707 |
| 17 | 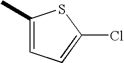 | 727 |
| 18 | 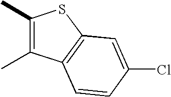 | 791 |
| 19 | 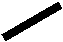 | 625 |
| 20 | 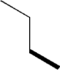 | 653 |
| 21 | 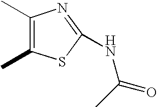 | 765 |
| 22 | 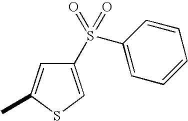 | 833 |
| 23 | 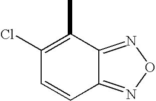 | 763 |
| 24 | 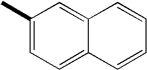 | 737 |
| 25 | 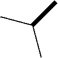 | 652 |

TABLE 3

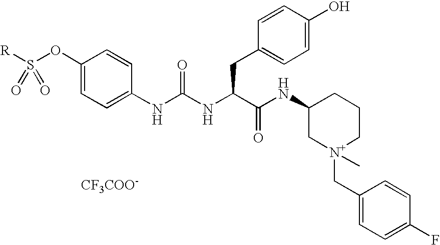

| Example | R | MS [M]⁺ |
|---|---|---|
| 26 | 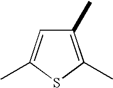 | 695 |
| 27 | 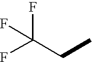 | 667 |
| 28 | 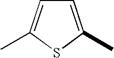 | 681 |
| 29 | 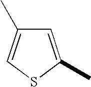 | 681 |
| 30 | 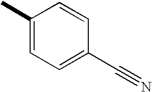 | 686 |
| 31 | 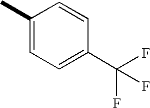 | 729 |
| 32 | 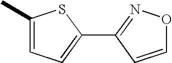 | 734 |
| 33 | 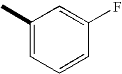 | 679 |
| 34 | 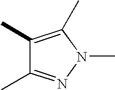 | 693 |
| 35 | 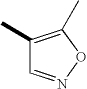 | 666 |
| 36 | 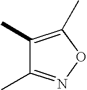 | 680 |
| 37 | 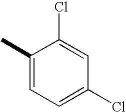 | 729 |
| 38 | 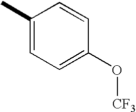 | 745 |
| 39 | 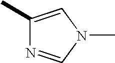 | 665 |
| 40 | 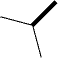 | 627 |

Proceeding in a similar manner as described in example 1, but replacing sulfonyl chloride with acid chloride and/or replacing 3,4-bis(methyloxy)benzaldehyde with the appropriate aldehydes, the compounds listed in Tables 4-5 were prepared.

TABLE 4

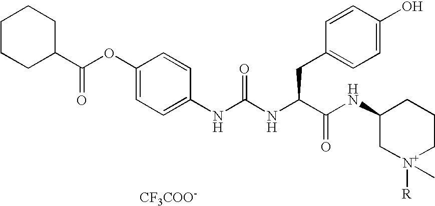

| Example | R | MS [M]⁺ |
|---|---|---|
| 41 | 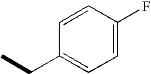 | 631 |
| 42 | 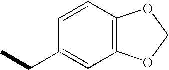 | 656 |
| 43 | 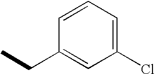 | 646 |
| 44 | 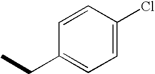 | 646 |
| 45 | 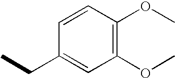 | 672 |

TABLE 5

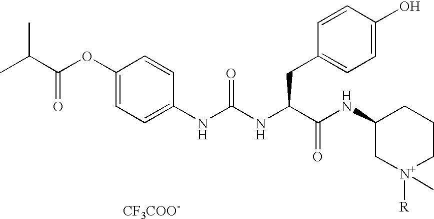

| Example | R | MS [M]⁺ |
|---|---|---|
| 46 | 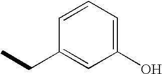 | 589 |
| 47 | 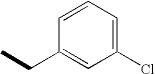 | 607 |
| 48 | 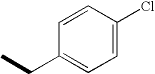 | 607 |

Preparation 2

4-Nitrobenzene sulfonyl chloride reacted with isopropyl amine to provide the isopropyl sulfonyl amide 9. The nitro group in 9 was converted to amine 10 via SnCl2. The amine was coupled with resin-bound amines 4 to afford the corresponding resin-bound ureas 11. The urea was subsequently treated with benzenethiolate to give secondary amine, which underwent reductive amination with appropriate aldehydes to produce tertiary amine 12. Amine 12 was then treated with alkyl halides to form the corresponding resin-bound quaternary ammonium salts, which were cleaved with 50% trifluoroacetic acid in dichloromethane to afford targeted compounds 13. 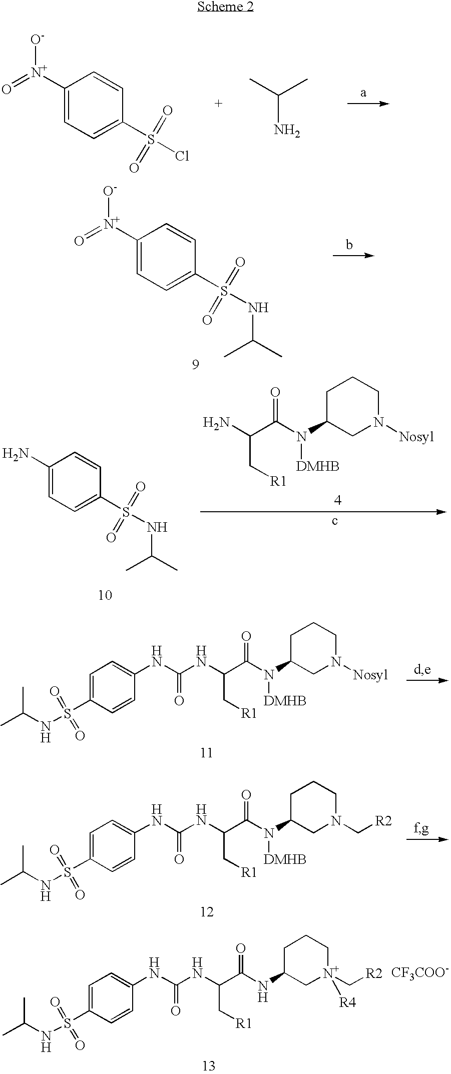
Conditions: a) Toluene 80° C. b) SnCl2, EtOH, 70° C.; c) 4-nitrobenzene chloroformate, tetrahydrofuran, diisopropylethylamine, dimethyl formamide, rt; d) K₂CO₃, PhSH, 1-methyl-2-pyrrolidinone, rt; e) R2CHO, Na(OAc)₃BH, 10% acetic acid in 1-methyl-2-pyrrolidinone, rt; f) R4Z, acetonitrile, rt; g) 50% trifluoroacetic acid in dichloromethane, rt.

The following examples are provided as illustrative of the present invention but not limiting in any way:

### Example 49

### Preparation of N-{(3S)-1-[(3-hydroxyphenyl)methyl]-1-methyl-3-piperidiniumyl}-N-{[(4-{[(1-methylethyl)amino]sulfonyl}phenyl)amino]carbonyl}-L-tyrosinamide Trifluoroacetate

A solution of 4-nitrobezenesulfonyl chloride (2000 mg, 9.05 mmol) in 20 mL toluene was added dropwise to a solution of isopropylamine (1067 mg, 18.1 mmol) in 50 mL toluene. The reaction mixture was heated to 80° C. for 1 hour and cooled to room temperature. Water (25 mL) was added. The aqueous phase was extracted with ethyl acetate (3×50 mL). The combined organic phase was dried over MgSO₄, concentrated and run through a pad of silica gel eluting with hexane:ethyl acetate (1:1) to give amide (1800 mg, 93%). MS (ESI) 245 [M+H]⁺.

To Isopropyl amide 1400 mg, 5.71 mmol) in 200 mL ethyl alcohol was added SnCl₂ (5420 mg, 28.6 mmol). The reaction mixture was stirred at 70° C. for 3 hours. The reaction mixture was concentrated. Ethyl acetate 100 ml) and saturated NaHCO₃ (60 mL) were added. Sn salts was precipitated out after 30 mins and filtered off. The organic phase was washed with brine, dried over MgSO₄ and concentrated to afford amine 10(1100 mg, 90%). MS (ESI) 215 [M+H]⁺.

To a mixture of 196 mg (0.8 mmol) of 4-amino-N-(1-methylethyl)benzenesulfonamide in 3 ml anhydrous tetrahydrofuran was added 169 mg (0.84 mmol) 4-nitrobenzenechloroformate. The reaction mixture was stirred at room temperature for half an hour and concentrated. Diisopropylethylamine (0.28 mL, 1.6 mmol), DMHB resin bound O-(1,1-dimethylethyl)-N-{(3S)-1-[(2-nitrophenyl)sulfonyl]-3-pyrrolidinyl}-L-tyrosinamide 4 (400 mg, 0.32 mmol) and dimethyl formamide (5 mL) were added to reaction mixture and shaked overnight. The resin was washed with DMF (3×10 mL), CH₂Cl₂/MeOH (1:1, 3×10 mL) and MeOH (3×10 mL). The resulting urea resin 11 was dried in vacuum oven at 35° C. for 24 h. An analytical amount of resin was cleaved with 50% trifluoroacetic acid in dichloromethane for 2 h at rt. The resulting solution was concentrated in vacuo: MS (ESI) 689.6 [M+H-tBu]⁺.

To a mixture of urea resin 11 (0.32 mmol) in 4 mL of 1-methyl-2-pyrrolidinone was added 332 mg (2.4 mmol) of K₂CO₃ and 0.12 mL (1.6 mmol) of PhSH. After the resulting mixture was shaken at rt for 2 h, the resin was washed with DMF (3×10 mL), H₂O (3×10 mL), DMF (3×10 mL), CH₂Cl₂/MeOH (1:1, 3×10 mL) and MeOH (3×10 mL). The resulting resin was dried in vacuum oven at 35° C. for 24 h. The resulting solution was concentrated in vacuo: MS (ESI) 504.4 [M+H-tBu]⁺.

To a mixture of the above dry resin secondary amine (0.16 mmol) in 4 mL of 10% HOAc in anhydrous 1-methyl-2-pyrrolidinone solution was added 292.8 mg (2.4 mmol) of 3-hydroxylbenzaldehyde and 508.8 mg (2.4 mmol) of sodium triacetoxyborohydride. After the resulting mixture was shaken at rt for 24 h, the resin was washed with DMF (3×10 mL), CH₂Cl₂/MeOH (1:1, 3×10 mL) and MeOH (3×10 mL). The resulting resin 12 was dried in vacuum oven at 35° C. for 24 h. An analytical amount of resin was cleaved with 50% trifluoroacetic acid in dichloroethane for 2 h at rt. The resulting solution was concentrated in vacuo: MS (ESI) 610.4 [M+H-tBu]⁺.

To a mixture of resin-bound tertiary amines 12 ( 0.16 mmol) in 4 mL of anhydrous acetonitrile was added 74 μL (1.2 mmol) of iodomethane. After the mixture was shaken at rt for 16 h, the resin was washed with DMF (3×10 mL), CH₂Cl₂/MeOH (1:1, 3×10 mL), MeOH (3×10 mL) and CH₂Cl₂ (3×10 mL). The resulting resin was dried in vacuum oven at 35° C. for 24 h. The dry resin was treated with 4 mL of 50% trifluoroacetic acid in dichloroethane at rt for 2 h. After the cleavage solution was collected, the resin was treated with another 4 mL of 50% trifluoroacetic acid in dichloroethane at rt for 10 min. The combined cleavage solutions were concentrated in vacuo. The residue was purified using a Gilson semi-preparative HPLC system with a YMC ODS-A (C-18) column 50 mm by 20 mm ID, eluting with 10% B to 90% B in 3.2 min, hold for 1 min where A=H₂O (0.1% trifluoroacetic acid) and B=CH₃CN (0.1% trifluoroacetic acid) pumped at 25 mL/min, to produce of N-{(3S)-1-[(3-hydroxyphenyl)methyl]-1-methyl-3-piperidiniumyl}-N-{[(4-{[(1-methylethyl)amino]sulfonyl}phenyl)amino]carbonyl}-L-tyrosinamide_trifluoroacetate (white powder, 50 mg, 50% over 5 steps): MS (ESI) 624 [M]⁺.

Proceeding in a similar manner as described in example 49, but replacing 3-hydroxyl benzaldehyde with 1,3-benzodioxole-5-carbaldehyde, the compound listed in Tables 6 was prepared.

TABLE 6

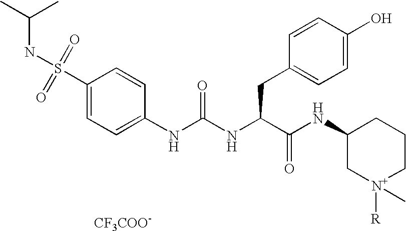

| Example | R | MS [M]⁺ |
|---|---|---|
| 50 | 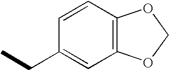 | 653 |

Proceeding in a similar manner as described in example 1, but replacing 1,1-dimethylethyl (3S)-3-piperidinylcarbamate with 1,1-dimethylethyl (3S)-3-pyrrolidinylcarbamate, and/or replacing 3,4-bis(methyloxy)benzaldehyde 3-hydroxyl benzaldehyde, and replacing methyl iodide with ethyl iodide to make the quaternary ammonium salt, the compound listed in Tables 7 was prepared.

TABLE 7

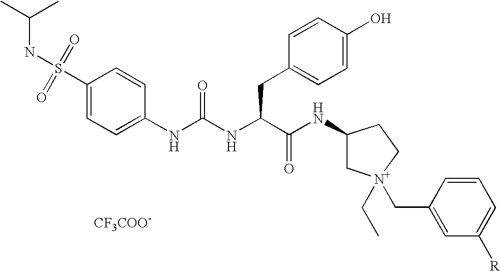

| Example | R | MS [M]⁺ |
|---|---|---|
| 51 | OH | 624 |

### BIOLOGICAL EXAMPLES

The inhibitory effects of compounds at the M₃ mAChR of the present invention are determined by the following in vitro and in vivo assays:

Analysis of Inhibition of Receptor Activation by Calcium Mobilization:

1) 384-Well FLIPR Assay

A CHO (chinese hamster ovary) cell line stably expressing the human M3 muscarinic acetylcholine receptor is grown in DMEM plus 10% FBS, 2 mM Glutamine and 200 ug/ml G418. Cells are detached for maintenance and for plating in preparation for assays using either enzymatic or ion chelation methods. The day before the FLIPR (fluorometric imaging plate reader) assay, cells are detached, resuspended, counted, and plated to give 20,000 cells per 384 well in a 50 ul volume. The assay plates are black clear bottom plates, Becton Dickinson catalog number 35 3962. After overnight incubation of plated cells at 37 degrees C. in a tissue culture incubator, the assay is run the next day. To run the assay, media are aspirated, and cells are washed with 1× assay buffer (145 mM NaCl, 2.5 mM KCl, 10 mM glucose, 10 mM HEPES, 1.2 mM MgCl₂, 2.5 mM CaCl₂, 2.5 mM probenecid (pH 7.4.) Cells are then incubated with 50 ul of Fluo-3 dye (4 uM in assay buffer) for 60-90 minutes at 37 degrees C. The calcium-sensitive dye allows cells to exhibit an increase in fluorescence upon response to ligand via release of calcium from intracellular calcium stores. Cells are washed with assay buffer, and then resuspended in 50 ul assay buffer prior to use for experiments. Test compounds and antagonists are added in 25 ul volume, and plates are incubated at 37 degrees C. for 5-30 minutes. A second addition is then made to each well, this time with the agonist challenge, acetylcholine. It is added in 25 ul volume on the FLIPR instrument. Calcium responses are measured by changes in fluorescent units. To measure the activity of inhibitors/antagonists, acetylcholine ligand is added at an EC₈₀ concentration, and the antagonist IC₅₀ can then be determined using dose response dilution curves. The control antagonist used with M3 is atropine.

2) 96-Well FLIPR Assay

Stimulation of mAChRs expressed on CHO cells were analyzed by monitoring receptor-activated calcium mobilization as previously described. CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl2, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels. The emitted fluorescence from all 96 wells is measured simultaneously using a cooled CCD camera. Data points are collected every second. This data was then plotting and analyzed using GraphPad PRISM software.

Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine. Mice were pretreated with 50 μl of compound (0.003-10 μg/mouse) in 50 μl of vehicle (10% DMSO) intranasally, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A compound of formula I as indicated below: 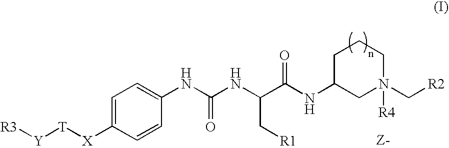
wherein
n is 0 or 1;
When X is nitrogen or oxygen, Y is nothing;
When Y is nitrogen or oxygen, X is nothing;
T is a sulfonyl group (SO2) or canbonyl group (CO);
When T=CO, X is oxygen or nitrogen;
Z⁻ is selected from the group consisting of halo, CF3COO⁻, mesylate, tosylate, or any other pharmaceutically acceptable counter ion;
R1 is selected from the group consisting of C₁-C₈ branched or unbranched alkyl, C₃-C₈ cycloalkyl, C₃-C₈ cycloalkyl lower alkyl, C₃-C₈ alkenyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl, C₃-C₈ cycloalkyl lower alkyl, phenyl and phenyl C1-C3 lower alkyl.
R2 is selected from the group consisting of C₁-C₈ branched or unbranched alkyl, C₃-C₈ cycloalkyl, C₃-C₈ cycloalkyl lower alkyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl and C₃-C₈ cycloalkyl lower alkyl and heterocycle rings;
R3 is selected from the group consisting of an unsubstituted or substituted following group: phenyl, phenyl C1-C6 lower alkyl, thiophenyl, thiophenyl C1-C6 lower alkyl, furanyl, furanyl C1-C6 lower alkyl, pyridinyl, pyridinyl C1-C6 lower alkyl, imidazolyl, imidazolyl C1-C6 lower alkyl, naphthyl, naphthyl C1-C6 lower alkyl, quinolinyl, quinolinyl C1-C6 lower alkyl, indolyl, indolyl C1-C6 lower alkyl, benzothiophenyl, benzothiophenyl C1-C6 lower alkyl, benzofuranyl, benzofuranyl C1-C6 lower alkyl, benzoimidazolyl, benzoimidazolyl C1-C6 lower alkyl, C₁-C₈ branched or unbranched alkyl, C₃-C₈ cycloalkyl, C₃-C₈ cycloalkyl C₁-C₆ lower alkyl, or C₃-C₈ alkenyl; wherein, when substituted, a group is substituted by one or more radicals selected from the group consisting of C₁-C₈ alkoxy, phenoxy, phenyl C₁-C₃ alkoxy, halo, hydroxy, amino, cyano, trifluoromethyl, methylenedioxy, ethylenedioxy, propylenedioxy, butylenedioxy, C₁-C₈ branched or unbranched alkyl, C₃-C₈ cycloalkyl, C₃-C₈ cycloalkyl lower alkyl, phenyl, phenyl C1-C3 lower alkyl, thiophenyl, thiophenyl C1-C3 lower alkyl, furanyl, furanyl C1-C3 lower alkyl, pyridinyl, pyridinyl C1-C3 lower alkyl, naphthyl, naphthyl C1-C3 lower alkyl, quinolinyl, quinolinyl C1-C3 lower alkyl, indolyl, indolyl C1-C3 lower alkyl, benzothiophenyl, benzothiophenyl C1-C3 lower alkyl, benzofuranyl, benzofuranyl C1-C3 lower alkyl, COOH, COR6, COOR6, CONHR6, CON(R6)2, COG, NHR6, N(R6)2, G, OCOR6, OCONHR6, NHCOR6, N(R6)COR6, NHCOOR6 and NHCONHR6;
R4 is selected from the group consisting of C₁-C₈ branched or unbranched alkyl, C₃-C₈ cycloalkyl, C₃-C₈ cycloalkyl lower alkyl.

2. A compound according to claim 1 selected from the group consisting of:
n is 0 or 1;
When X is nitrogen or oxygen, Y is nothing;
When Y is nitrogen or oxygen, X is nothing;
T is a sulfonyl group (SO2) or canbonyl group (CO);
When T=CO, X is oxygen or nitrogen;
Z⁻ is selected from the group consisting of halo, CF3COO⁻, mesylate, tosylate, or any other pharmaceutically acceptable counter ion;
R1 is selected from the group consisting of C1-C8 branched or unbranched alkyl, C₃-C₈ cycloalkyl, C₃-C₈ cycloalkyl lower alkyl, C₃-C₈ alkenyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl, C₃-C₈ cycloalkyl lower alkyl, phenyl and phenyl C1-C3 lower alkyl.
R2 is selected from the group consisting of C₁-C₈ branched or unbranched alkyl, C₃-C₈ cycloalkyl, C₃-C₈ cycloalkyl lower alkyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl and C₃-C₈ cycloalkyl lower alkyl and heterocycle rings;
R3 is selected from the group consisting of an unsubstituted or substituted following group: phenyl, phenyl C1-C6 lower alkyl, thiophenyl, thiophenyl C1-C6 lower alkyl, furanyl, furanyl C1-C6 lower alkyl, pyridinyl, pyridinyl C1-C6 lower alkyl, imidazolyl, imidazolyl C1-C6 lower alkyl, naphthyl, naphthyl C1-C6 lower alkyl, quinolinyl, quinolinyl C1-C6 lower alkyl, indolyl, indolyl C1-C6 lower alkyl, benzothiophenyl, benzothiophenyl C1-C6 lower alkyl, benzofuranyl, benzofuranyl C1-C6 lower alkyl, benzoimidazolyl, benzoimidazolyl C1-C6 lower alkyl, C1-C8 branched or unbranched alkyl, C₃-C₈ cycloalkyl, C₃-C₈ cycloalkyl C₁-C₆ lower alkyl, or C₃-C₈ alkenyl; wherein, when substituted, a group is substituted by one or more radicals selected from the group consisting of C₁-C₈ alkoxy, phenoxy, phenyl C₁-C₃ alkoxy, halo, hydroxy, amino, cyano, trifluoromethyl, methylenedioxy, ethylenedioxy, propylenedioxy, butylenedioxy, C₁-C₈ branched or unbranched alkyl, C₃-C₈ cycloalkyl, C₃-C₈ cycloalkyl lower alkyl, phenyl, phenyl C1-C3 lower alkyl, thiophenyl, thiophenyl C1-C3 lower alkyl, furanyl, furanyl C1-C3 lower alkyl, pyridinyl, pyridinyl C1-C3 lower alkyl, naphthyl, naphthyl C1-C3 lower alkyl, quinolinyl, quinolinyl C1-C3 lower alkyl, indolyl, indolyl C1-C3 lower alkyl, benzothiophenyl, benzothiophenyl C1-C3 lower alkyl, benzofuranyl, benzofuranyl C1-C3 lower alkyl, COOH, COR6, COOR6, CONHR6, CON(R6)2, COG, NHR6, N(R6)2, G, OCOR6, OCONHR6, NHCOR6, N(R6)COR6, NHCOOR6 and NHCONHR6;
R4 is selected from the group consisting of C₁-C₈ branched or unbranched alkyl, C₃-C₈ cycloalkyl, C₃-C₈ cycloalkyl lower alkyl.
or a pharmaceutically acceptable salt thereof.

3. A compound according to claim 1 selected from the group consisting of:
n is 0 or 1;
When X is nitrogen or oxygen, Y is nothing;
When Y is nitrogen or oxygen, X is nothing;
T is a sulfonyl group (SO2) or canbonyl group (CO);
When T=CO, X is oxygen or nitrogen;
Z⁻ is selected from the group consisting of halo, CF3COO⁻, mesylate, tosylate, or any other pharmaceutically acceptable counter ion;
R1 is selected from the group consisting of C₁-C₈ branched or unbranched alkyl, C₃-C₈ cycloalkyl, C₃-C₈ cycloalkyl lower alkyl, C₃-C₈ alkenyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl, C₃-C₈ cycloalkyl lower alkyl, phenyl and phenyl C1-C3 lower alkyl.
R2 is selected from the group consisting of C₁-C₈ branched or unbranched alkyl, C₃-C₈ cycloalkyl, C₃-C₈ cycloalkyl lower alkyl, unsubstituted or substituted phenyl, or unsubstituted or substituted phenyl C1-C3 lower alkyl; wherein, when substituted, a group is substituted by one or more radicals selected from the group consisting of C₁-C₈ alkoxy, halo, hydroxy, amino, cyano, trifluoromethyl, C₁-C₈ branched or unbranched alkyl, C₃-C₈ cycloalkyl and C₃-C₈ cycloalkyl lower alkyl and heterocycle rings;
R3 is selected from the group consisting of an unsubstituted or substituted following group: phenyl, phenyl C1-C6 lower alkyl, thiophenyl, thiophenyl C1-C6 lower alkyl, furanyl, furanyl C1-C6 lower alkyl, pyridinyl, pyridinyl C1-C6 lower alkyl, imidazolyl, imidazolyl C1-C6 lower alkyl, naphthyl, naphthyl C1-C6 lower alkyl, quinolinyl, quinolinyl C1-C6 lower alkyl, indolyl, indolyl C1-C6 lower alkyl, benzothiophenyl, benzothiophenyl C1-C6 lower alkyl, benzofuranyl, benzofuranyl C1-C6 lower alkyl, benzoimidazolyl, benzoimidazolyl C1-C6 lower alkyl, C₁-C₈ branched or unbranched alkyl, C₃-C₈ cycloalkyl, C₃-C₈ cycloalkyl C₁-C₆ lower alkyl, or C₃-C₈ alkenyl; wherein, when substituted, a group is substituted by one or more radicals selected from the group consisting of C₁-C₈ alkoxy, phenoxy, phenyl C₁-C₃ alkoxy, halo, hydroxy, amino, cyano, trifluoromethyl, methylenedioxy, ethylenedioxy, propylenedioxy, butylenedioxy, C₁-C₈ branched or unbranched alkyl, C₃-C₈ cycloalkyl, C₃-C₈ cycloalkyl lower alkyl, phenyl, phenyl C1-C3 lower alkyl, thiophenyl, thiophenyl C1-C3 lower alkyl, furanyl, furanyl C1-C3 lower alkyl, pyridinyl, pyridinyl C1-C3 lower alkyl, naphthyl, naphthyl C1-C3 lower alkyl, quinolinyl, quinolinyl C1-C3 lower alkyl, indolyl, indolyl C1-C3 lower alkyl, benzothiophenyl, benzothiophenyl C1-C3 lower alkyl, benzofuranyl, benzofuranyl C1-C3 lower alkyl, COOH, COR6, COOR6, CONHR6, CON(R6)2, COG, NHR6, N(R6)2, G, OCOR6, OCONHR6, NHCOR6, N(R6)COR6, NHCOOR6 and NHCONHR6;
R4 is selected from the group consisting of C₁-C₈ branched or unbranched alkyl, C₃-C₈ cycloalkyl, C₃-C₈ cycloalkyl lower alkyl.

4. or a pharmaceutically acceptable salt thereof.

4. A compound according to claim 1 selected from the group consisting of:
N-((3S)-1-{[3,4-bis(methyloxy)phenyl]methyl}-1-methyl-3-piperidiniumyl)-N-{[(4-{[(2,2,2-trifluoroethyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(5-methyl-2-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(4-methyl-2-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-[({4-[(8-quinolinylsulfonyl)oxy]phenyl}amino)carbonyl]-L-tyrosinamide trifluoroacetate;
N-((3S)-1-{[3,4-bis(methyloxy)phenyl]methyl}-1-methyl-3-piperidiniumyl)-N-({[4-({[3,4-bis(methyloxy)phenyl]sulfonyl}oxy)phenyl]amino}carbonyl)-L-tyrosinamide trifluoroacetate;
N-((3S)-1-{[3,4-bis(methyloxy)phenyl]methyl}-1-methyl-3-piperidiniumyl)-N-{[(4-{[(2-bromophenyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(4-fluorophenyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-[({4-[(phenylsulfonyl)oxy]phenyl}amino)carbonyl]-L-tyrosinamide trifluoroacetate;
N-((3S)-1-{[3,4-bis(methyloxy)phenyl]methyl}-1-methyl-3-piperidiniumyl)-N-{[(4-{[(5-bromo-2-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-[({4-[(3-thienylsulfonyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-{[(4-{[(2,5-dimethyl-3-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(2,2,2-trifluoroethyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(5-methyl-2-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(4-methyl-2-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(5-chloro-2-thienyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-[({4-[(methylsulfonyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-[({4-[(propylsulfonyl)oxy]phenyl}amino)carbonyl]-L-tyrosinamide trifluoroacetate;
N-({[4-({[2-(acetylamino)-4-methyl-1,3-thiazol-5-yl]sulfonyl}oxy)phenyl]amino}carbonyl)-N-[(3S)-1-(1,3-benzodioxol-5-ylmethyl)-1-methyl-3-piperidiniumyl]-L-tyrosinamide trifluoroacetate;
N-[(3S)-1-(1,3-benzodioxol-5-ylmethyl)-1-methyl-3-piperidiniumyl]-N-({[4-({[4-(phenylsulfonyl)-2-thienyl]sulfonyl}oxy)phenyl]amino}carbonyl)-L-tyrosinamide trifluoroacetate;
N-[(3S)-1-(1,3-benzodioxol-5-ylmethyl)-1-methyl-3-piperidiniumyl]-N-{[(4-{[(5-chloro-2,1,3-benzoxadiazol-4-yl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-[({4-[(2-naphthalenylsulfonyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-{[(4-{[(2,2,2-trifluoroethyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(5-methyl-2-thienyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(4-methyl-2-thienyl)sulfonyl]oxy}phenyl)amino]carbonyl}-L-tyrosinamide trifluoroacetate;
N-{[(4-{[(4-cyanophenyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{(3S)-1-[(4-fluorophenyl)methyl]-1-methyl-3-piperidiniumyl}-N-({[4-({[4-(trifluoromethyl)phenyl]sulfonyl}oxy)phenyl]amino}carbonyl)-L-tyrosinamide trifluoroacetate;
N-{(3S)-1-[(4-fluorophenyl)methyl]-1-methyl-3-piperidiniumyl}-N-({[4-({[5-(3-isoxazolyl)-2-thienyl]sulfonyl}oxy)phenyl]amino}carbonyl)-L-tyrosinamide trifluoroacetate;
N-{(3S)-1-[(4-fluorophenyl)methyl]-1-methyl-3-piperidiniumyl}-N-{[(4-{[(3-fluorophenyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(1,3,5-trimethyl-1H-pyrazol-4-yl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(5-methyl-4-isoxazolyl)sulfonyl]oxy}phenyl)amino]carbonyl}-L-tyrosinamide trifluoroacetate;
N-{[(4-{[(3,5-dimethyl-4-isoxazolyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{[(4-{[(2,4-dichlorophenyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{(3S)-1-[(4-fluorophenyl)methyl]-1-methyl-3-piperidiniumyl}-N-[({4-[({4-[(trifluoromethyl)oxy]phenyl}sulfonyl)oxy]phenyl}amino)carbonyl]-L-tyrosinamide trifluoroacetate;
N-{(3S)-1-[(4-fluorophenyl)methyl]-1-methyl-3-piperidiniumyl}-N-{[(4-{[(1-methyl-1H-imidazol-4-yl)sulfonyl]oxy}phenyl)amino]carbonyl}-L-tyrosinamide trifluoroacetate;
N-[({4-[(cyclohexylcarbonyl)oxy]phenyl}amino)carbonyl]-N-{(3S)-1-[(4-fluorophenyl)methyl]-1-methyl-3-piperidiniumyl}-L-tyrosinamide trifluoroacetate;
N-[(3S)-1-(1,3-benzodioxol-5-ylmethyl)-1-methyl-3-piperidiniumyl]-N-[({4-[(cyclohexylcarbonyl)oxy]phenyl}amino)carbonyl]-L-tyrosinamide trifluoroacetate;
N-{(3S)-1-[(4-chlorophenyl)methyl]-1-methyl-3-piperidiniumyl}-N-[({4-[(cyclohexylcarbonyl)oxy]phenyl}amino)carbonyl]-L-tyrosinamide trifluoroacetate;
N-{(3S)-1-[(3-chlorophenyl)methyl]-1-methyl-3-piperidiniumyl}-N-[({4-[(cyclohexylcarbonyl)oxy]phenyl}amino)carbonyl]-L-tyrosinamide trifluoroacetate;
N-((3S)-1-{[3,4-bis(methyloxy)phenyl]methyl}-1-methyl-3-piperidiniumyl)-N-[({4-[(cyclohexylcarbonyl)oxy]phenyl}amino)carbonyl]-L-tyrosinamide trifluoroacetate;
N-{(3S)-1-[(3-hydroxyphenyl)methyl]-1-methyl-3-piperidiniumyl}-N-[({4-[(2-methylpropanoyl)oxy]phenyl}amino)carbonyl]-L-tyrosinamide trifluoroacetate;
N-{(3S)-1-[(3-chlorophenyl)methyl]-1-methyl-3-piperidiniumyl}-N-[({4-[(2-methylpropanoyl)oxy]phenyl}amino)carbonyl]-L-tyrosinamide trifluoroacetate;
N-{(3S)-1-[(4-chlorophenyl)methyl]-1-methyl-3-piperidiniumyl}-N-[({4-[(2-methylpropanoyl)oxy]phenyl}amino)carbonyl]-L-tyrosinamide trifluoroacetate;
N-[(3S)-1-(1,3-benzodioxol-5-ylmethyl)-1-methyl-3-piperidiniumyl]-N-{[(4-{[(1-methylethyl)amino]sulfonyl}phenyl)amino]carbonyl}-L-tyrosinamide trifluoroacetate;
N-{(3S)-1-ethyl-1-[(3-hydroxyphenyl)methyl]-3-pyrrolidiniumyl}-N-{[(4-{[(1-methylethyl)amino]sulfonyl}phenyl)amino]carbonyl}-L-tyrosinamide trifluoroacetate;
or any other pharmaceutically acceptable salt.

5. A compound according to claim 1 selected from the group consisting of:
N-((3S)-1-{[3,4-bis(methyloxy)phenyl]methyl}-1-methyl-3-piperidiniumyl)-N-{[(4-{[(2,5-dimethyl-3-thienyl)sulfonyl]oxy}phenyl)amino]carbonyl}-L-tyrosinamide trifluoroacetate
N-((3S)-1-{[3,4-bis(methyloxy)phenyl]methyl}-1-methyl-3-piperidiniumyl)-N-{[(4-{[(2,5-dimethyl-3-thienyl)sulfonyl]oxy}phenyl)amino]carbonyl}-L-tyrosinamide trifluoroacetate;
N-((3S)-1-{[3,4-bis(methyloxy)phenyl]methyl}-1-methyl-3-piperidiniumyl)-N-{[(4-{[(1-methylethyl)sulfonyl]oxy}phenyl)amino]carbonyl}-L-tyrosinamide trifluoroacetate;
N-[(3S)-1-(1,3-benzodioxol-5-ylmethyl)-1-methyl-3-piperidiniumyl]-N-{[(4-{[(6-chloro-3-methyl-1-benzothien-2-yl) sulfonyl]oxy}phenyl)amino]carbonyl}-L-tyrosinamide trifluoroacetate;
N-{[(4-{[(2,5-dimethyl-3-thienyl)sulfonyl]oxy}phenyl)amino]carbonyl}-N-{(3S)-1-[(4-fluorophenyl)methyl]-1-methyl-3-piperidiniumyl}-L-tyrosinamide trifluoroacetate;
N-{(3S)-1-[(4-fluorophenyl)methyl]-1-methyl-3-piperidiniumyl}-N-{[(4-{[(1-methylethyl)sulfonyl]oxy}phenyl)amino]carbonyl}-L-tyrosinamide trifluoroacetate;
N-{(3S)-1-[(4-fluorophenyl)methyl]-1-methyl-3-piperidiniumyl}-N-{[(4-{[(1-methylethyl)sulfonyl]oxy}phenyl)amino]carbonyl}-L-tyrosinamide trifluoroacetate;
N-{(3S)-1-[(3-hydroxyphenyl)methyl]-1-methyl-3-piperidiniumyl}-N-{[(4-{[(1-methylethyl)amino]sulfonyl}phenyl)amino]carbonyl}-L-tyrosinamide trifluoroacetate
or any other pharmaceutically acceptable salt, or non-salt form thereof.

6. A Pharmaceutical composition for the treatment of muscarinic acetylcholine receptor mediated diseases comprising a compound according to claim 1 and a pharmaceutically acceptable carrier thereof.

7. A method of inhibiting the binding of acetylcholine to its receptors in a mammal in need thereof comprising administering a safe and effective amount of a compound according to claim 1.

8. A method of treating a muscarinic acetylcholine receptor mediated disease, wherein acetylcholine binds to said receptor, comprising administering a safe and effective amount of a compound according to claim 1.

9. A method according to claim 8 wherein the disease is selected from the group consisting of chronic obstructive lung disease, chronic bronchitis, asthma, chronic respiratory obstruction, pulmonary fibrosis, pulmonary emphysema and allergic rhinitis.

10. A method according to claim 9 wherein administration is via inhalation via the mouth or nose.

11. A method according to claim 10 wherein administration is via a medicament dispenser selected from a reservoir dry powder inhaler, a multi-dose dry powder inhaler or a metered dose inhaler.

12. A method according to claim 11 wherein the compound is administered to a human and has a duration of action of 12 hours or more for a 1 mg dose.

13. A method according to claim 12 wherein the compound has a duration of action of 24 hours or more.

14. A method according to claim 13 wherein the compound has a duration of action of 36 hours or more.
